(19) United States
(12) Patent Application Publication
(10) Pub. No.: US 2006/0235416 A1
(43) Pub. Date: Oct. 19, 2006

(54) Intervertebral connecting elements

(75) Inventor: Paul Revis, Collierville, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/107,078

(22) Filed: Apr. 15, 2005

(51) Int. Cl. A61B 17/56

(52) U.S. Cl. 606/74

(57) ABSTRACT

A system for stabilizing a vertebral joint comprises a connection element adapted to extend through a first vertebral body, through a vertebral endplate of the first vertebral body, through a vertebral endplate of a second vertebral body, and through the second vertebral body.

### BACKGROUND

Severe back pain may be caused by injured, degraded, or diseased spinal joints and particularly spinal discs. Current methods of treating these damaged spinal discs may include vertebral fusion, nucleus replacements, or motion preservation disc prostheses. Despite treatment or even because of treatment, a patient may experience joint instability, particularly when the patient undertakes normal motions such as lateral bending and axial rotation. Thus, solutions are needed to overcome these and other problems that arise with the treatment of spinal joints.

### SUMMARY

According to one embodiment of this disclosure, a system for stabilizing a vertebral joint comprises a first connection element adapted to extend through a first pedicle of a first vertebra, through at least a portion of a vertebral body of the first vertebra, and into an endplate of a second vertebra. The system further comprises a second connection element adapted to extend through a second pedicle of a first vertebra, through at least a portion of the vertebral body of the first vertebra, and into the endplate of the second vertebra.

According to another embodiment of this disclosure, method of stabilizing a vertebral joint between first and second vertebrae comprises creating a first passage through a first pedicle of the first vertebra and into a vertebral body of the first vertebra. The method further comprises guiding a first connection element into the first passage and through the vertebral body of the first vertebra. The method further comprises guiding the first connection element into the second vertebra.

According to another embodiment of this disclosure, a system for stabilizing a vertebral joint comprises a means for connecting a first pedicle of a first vertebra to a vertebral body of a second vertebra and a means for connecting a second pedicle of the first vertebra to the vertebral body of the second vertebra.

According to another embodiment of this disclosure, A system for stabilizing a vertebral joint comprises a connection element adapted to extend through a first vertebral body, through a vertebral endplate of the first vertebral body, through a vertebral endplate of a second vertebral body, and through the second vertebral body.

### BRIEF DESCRIPTION OF THE DRAWINGS

FIG. 1 is a perspective view of a vertebral column having a damaged disc.

FIG. 2-7 are perspective views of different embodiments of a spinal joint repair system.

### DETAILED DESCRIPTION

The present disclosure relates generally to the field of orthopedic surgery, and more particularly to systems and methods for stabilizing a spinal joint. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the invention relates.

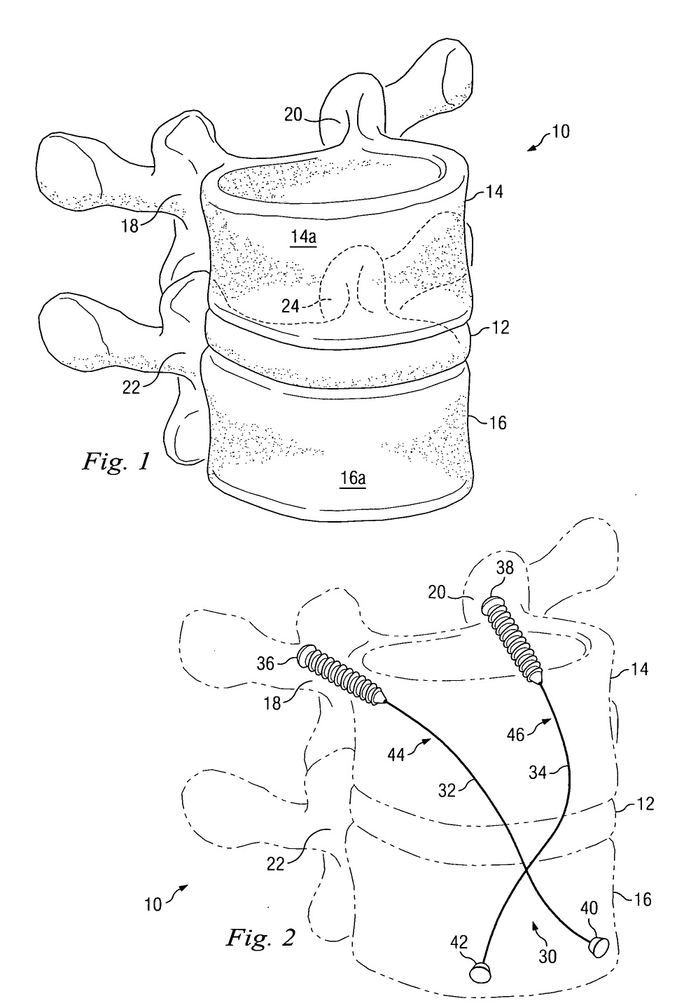

Referring first to FIG. 1, the numeral 10 refers to a vertebral joint which, in this example, includes an injured, diseased, or otherwise damaged intervertebral disc 12 extending between vertebrae 14, 16. The vertebra 14 includes pedicles 18, 20, and the vertebra 16 includes pedicles 22, 24. The vertebrae 14, 16 also include vertebral bodies 14a, 16a, respectively. A variety of surgical techniques, several of which are described below, may be used to repair the damaged spinal joint.

Referring now to FIG. 2, a vertebral joint stabilization system 30 may include connection elements 32, 34 which may be anchored to the pedicles of vertebra 14 with anchoring structures 36, 38, respectively. In this embodiment, the anchoring structures 36, 38 may be cannulated pedicle screws, but solid pedicle screws, suture anchors or other anchoring devices may be acceptable alternatives. The connection elements 32, 34 may be attached to the vertebra 16 with stopper features 40, 42, respectively, which may be for example, suture anchors, elastic plugs, or knots.

The connection elements may be formed of a biocompatible synthetic material or graft material such as autograft or allograft tendon material with bone attached to each end. The connection element material may, for example, take the form of a woven cable or unwoven strands. The connection elements may be adapted to withstand at least some tensile and/or torsional forces and may slacken when submitted to compressive forces. Additionally, the connection elements may be adapted to withstand at least some compressive and moment forces. Solid wire or rods, for example, may withstand such forces. These connection elements may permit some amount of flexibility and buckling when subject to compressive and/or moment loads.

To implant the system 30, an opening may be created in pedicle 18. A passage 44 may be formed from the opening through the pedicle 18, into the vertebral body of vertebra 14, and through the bottom endplate of the vertebra 14. The passage 44 may continue through the top endplate of the vertebra 16 and out through the anterior cortical wall of the vertebra 16. The passage 44 may extend diagonally through the spinal joint 10 such that the passage emerges through the anterior wall of the vertebra 16 on the side laterally opposite the pedicle 18. The cannulated pedicle screw 36 may be threaded into passage 44 in the pedicle 18. The connection element 32 may be anchored by the screw 36 and may extend through the passages in the vertebrae 14, 16. Where the connection element 32 passes through the outer wall of vertebra 16, the stopper feature 40 may be added to anchor the connection element 32 to the vertebra 16. When the patient is positioned in a neutral position, the connection element 32 may be relatively slack.

The passage 44 may be formed using a minimally invasive, flexible, steerable drill (not shown). The drill may be steered under radiographic guidance. In alternative embodiments, the passage may be created using a flexible stylet or another type of cutting or tunneling instrument. All or portions of the passage may even be formed by the connecting element itself. The passages through both vertebrae 14, 16 may be formed through a single approach from the pedicle 18. Alternatively, the passage through the upper vertebra 14 may be formed through the pedicle 18 and the passage through the lower vertebra 16 may be formed through a separate approach such as through an anterior opening in the lower vertebra 16.

The connecting element 34, the anchoring structure 38, and the stopper feature 42 may be implanted in a manner similar to the method described above for the implantation of connecting element 32. To implant these components of the system 30, an opening may be created in pedicle 20. A passage 46 may be formed from the opening through the pedicle 20, into the vertebral body of vertebra 14, and through the bottom endplate of the vertebra 14. The passage 46 may continue through the top endplate of the vertebra 16 and out through the anterior cortical wall of the vertebra 16. The passage 46 may extend diagonally through the spinal joint 10 such that the passage emerges through the anterior wall of the vertebra 16 on the side laterally opposite the pedicle 20. The cannulated pedicle screw 38 may be threaded into passage 46 in the pedicle 20. The connection element 34 may be anchored by the screw 38 and may extend through the passages in the vertebrae 14, 16. Where the connection element 34 passes through the outer wall of vertebra 16, the stopper feature 42 may be added to anchor the connection element 34 to the vertebra 16. When the patient is positioned in a neutral position, the connection element 34 may be relatively slack.

The diagonal paths taken by the passages 44, 46 may allow the connection elements 32, 34 to cross, however it is understood that the crossed paths may or may not intersect. In an alternative embodiment, the connection elements may extend from the pedicles of vertebra 14 and through the same lateral side of the anterior wall of the vertebra 16 as the pedicle of origin. Thus, the connection elements may remain on separate lateral sides of the joint 10 without crossing over one another.

The system 30 may be used restore or increase the range of joint stability. Specifically, the system 30 may provide lateral stability to the joint 10 by limiting lateral translation between the vertebrae 14, 16 and by limiting lateral bending. For example, lateral bending to the patient's right side may place the connection element 34 into tension, thereby limiting the right side lateral bending. Similarly, lateral translation of the vertebra 16 to the patient's right side, relative to the vertebra 14, may also place the connection element 34 into tension and limit the relative translation of the vertebrae. Additionally, the system 30 may be used to prevent spondylolisthesis between the vertebrae 14, 16. For example, as the vertebra 16 translates anteriorly relative to the vertebra 14, one or both connection elements 32, 34 may be placed into tension, thereby limiting the amount of anteroposterior displacement between the vertebrae. The system 30 may also provide axial stability by limiting axial rotation of the vertebra 14 relative to the vertebra 16. For example, axial rotation to the patient's right may place one or both of the connection elements 32, 34 into tension, thus restricting axial rotation.

It is understood that the connection elements may be selected to permit a predetermined amount of stability. For example the materials, the length, the amount of tautness/slack, or the method of fabrication of the connection elements may be selected to restrict motion based upon the anatomy of a particular patient. It is further understood that the materials, length, amount of tautness/slack, method of fabrication, or other factors may be different between the different connection elements, thereby permitting differing degrees of restraint. For example, a connection element corresponding to connection element 32 may be shorter than a connection element corresponding to connection element 34. Under this configuration, lateral bending to the patient's left side may be more restricted than lateral bending to the right.

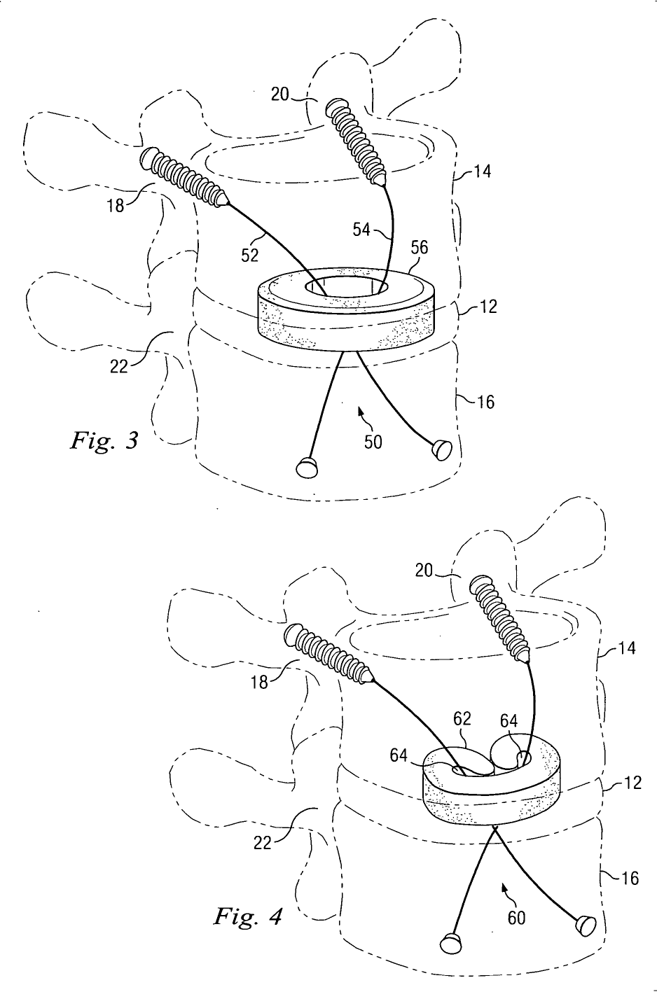

Referring now to FIG. 3, a vertebral joint stabilization system 50 may include connection elements 52, 54 which may be anchored to the pedicles of vertebra 14. The system 50 may be substantially similar to the system 20 with certain differences to be described below. In this embodiment, all or a portion of the damaged disc tissue including the nucleus of disc 12 may be excised. This procedure may be performed using an anterior, anterolateral, lateral, posterior, or other approach known to one skilled in the art. After this procedure, all or a portion of the annulus of the disc 12 may remain intact. The system 50 may further include a nucleus replacement 56 which may be inserted through an opening in the annulus to be held in place by the remnants of the natural annulus and/or by artificial annulus replacements. The nucleus replacement may be formed from a wide variety of natural or synthetic materials including biocompatible polymeric materials such as silicone, polyurethane, or hydrogels. More rigid materials including biocompatible metals and ceramics may also be appropriate for particular applications.

The connection elements 52, 54 may be implanted using a method similar to that described above for system 20. In this embodiment, however, the connection elements 52, 54 may extend through the nucleus replacement 56. The connection elements 52, 54 may provide the natural or increased stability described above with respect to system 20 and may additionally prevent migration or expulsion of the nucleus replacement 56.

In an alternative embodiment, the connection elements may anchor inside the vertebral body of vertebra 16, anchor directly to the upper endplate of vertebra 1, or anchor directly to the nucleus replacement.

The connection elements may be formed of a biocompatible synthetic material or graft material such as autograft or allograft tendon material with bone attached to each end. The connection element material may, for example, take the form of a woven cable or unwoven strands. The connection elements may be adapted to withstand at least some tensile and/or torsional forces and may slacken when submitted to compressive forces. Additionally, the connection elements may be adapted to withstand at least some compressive and moment forces. Solid wire or rods, for example, may withstand such forces. These connection elements may permit some amount of flexibility and buckling when subject to compressive and/or moment loads.

Referring now to FIG. 4, a vertebral joint stabilization system 60 may include a nucleus replacement 62. The system 60 may be substantially similar to system 50 with certain differences to be described below. The nucleus replacement 62 may be identical to or substantially similar to the implants described in U.S. Pat. No. 6,620,196 to Trieu, entitled “Intervertebral Disc Nucleus Implants and Methods,” which is incorporated by reference herein. In this embodiment, the nucleus replacement 62 may be held in a desired position by extending the connection elements through an aperture 64 in the replacement.

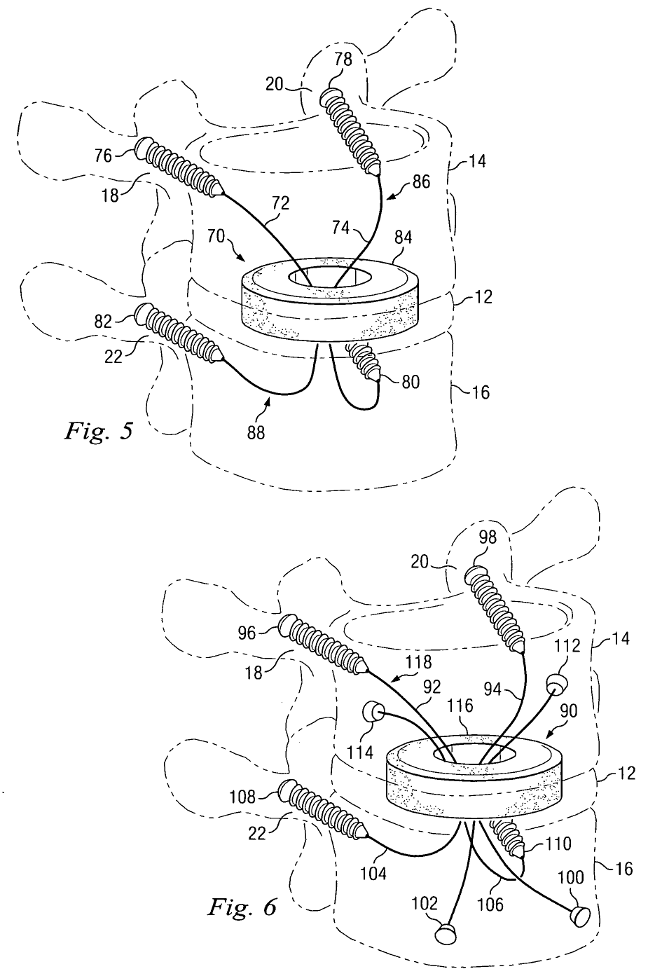

Referring now to FIG. 5, a vertebral joint stabilization system 70 may include connection elements 72, 74 which may be anchored to the pedicles of vertebra 14 with anchoring structures 76, 78, respectively, such as cannulated pedicle screws. Further, the connection elements 72, 74 may be attached to the pedicles of vertebra 16 with anchoring structures 80, 82, respectively, which may also be cannulated pedicle screws. The connection elements 72, 74 may extend through a nucleus replacement 84. The system 70 may be similar to system 50 with certain differences to be described.

The connection elements may be formed of a biocompatible synthetic material or graft material such as autograft or allograft tendon material with bone attached to each end. The connection element material may, for example, take the form of a woven cable or unwoven strands. The connection elements may be adapted to withstand at least some tensile and/or torsional forces and may slacken when submitted to compressive forces. Additionally, the connection elements may be adapted to withstand at least some compressive and moment forces. Solid wire or rods, for example, may withstand such forces. These connection elements may permit some amount of flexibility and buckling when subject to compressive and/or moment loads.

In this embodiment, all or a portion of the damaged tissue including the nucleus of disc 12 may be excised, and the nucleus replacement 84 installed as described above for system 50. Further, an opening may be created in the pedicle 20. A passage 86 may be formed from the opening, through the pedicle 20, into the vertebral body of vertebra 14, and through the bottom endplate of the vertebra 14. Additionally, an opening may be created in pedicle 22 and a passage 88 may be formed through the pedicle 22, into the vertebral body of vertebra 16, and through the upper endplate of vertebra 16. The cannulated pedicle screw 78 may be threaded into passage 86 in the pedicle 20. The connection element 74 may extend through the cannulated pedicle screw 78 and become anchored to the vertebra 14 by the pedicle screw. The connection element 74 may further extend through the passage 86, through the nucleus replacement 84, and through the passage 88. The cannulated pedicle screw 82 may be threaded into passage 88 in the pedicle 22, and the connection element 74 may be anchored to the vertebra 16 by the screw 82.

The passages 86, 88 may be formed using a flexible, steerable drill (not shown). The drill may be steered with radiographic guidance. In alternative embodiments, the passage may be created using a stylet or another type of cutting or tunneling instrument. All or portions of the passage may even be formed by the connecting element itself. The passages 86, 88 through the vertebrae 14, 16 may be formed using separate posterior approaches through pedicles 20, 22 as described. However, a single approach from one pedicle through to the second pedicle may be used in an alternative embodiment.

The connecting element 72 and the anchoring structures 76, 80 may be implanted in a manner similar to the method described above for the implantation of connecting element 74. The system 70 may provide the natural or increased stability described above with respect to system 50. Specifically, the system 70 may provide lateral stability to the joint 10 by limiting lateral translation between the vertebrae 14, 16 and by limiting lateral bending. The system 70 may also provide axial stability by limiting axial rotation of the vertebra 14 relative to the vertebra 16. Additionally, the connection elements 72, 74 extending through the nucleus replacement 84 may prevent migration or expulsion of the nucleus replacement.

Referring now to FIG. 6, a vertebral joint stabilization system 90 may include connection elements 92, 94 which may be anchored to the pedicles of vertebra 14 with anchoring structures 96, 98, respectively, such as cannulated pedicle screws. The connection elements 92, 94 may be attached to the vertebra 16 with stopper features 100, 102, respectively, which may be, for example, suture anchors, elastic plugs, or knots. The vertebral joint stabilization system 90 may further include connection elements 104, 106 which may be anchored to the pedicles of vertebra 16 with anchoring structures 108, 110, respectively, such as cannulated pedicle screws. The connection elements 104, 106 may be attached to the vertebra 14 with stopper features 112, 114, respectively, which may be, for example, suture anchors, elastic plugs, or knots. The connection elements 92, 94, 104, 106 may extend through a nucleus replacement 116. The connection elements 92, 94, 104, 106 and the nucleus replacement 116 may be formed from the materials as described above for elements 52, 54 and nucleus replacement 56, respectively.

The connection elements may be formed of a biocompatible synthetic material or graft material such as autograft or allograft tendon material with bone attached to each end. The connection element material may, for example, take the form of a woven cable or unwoven strands. The connection elements may be adapted to withstand at least some tensile and/or torsional forces and may slacken when submitted to compressive forces. Additionally, the connection elements may be adapted to withstand at least some compressive and moment forces. Solid wire or rods, for example, may withstand such forces. These connection elements may permit some amount of flexibility and buckling when subject to compressive and/or moment loads.

To implant the system 90, an opening may be created in pedicle 18. A passage 118 may be formed from the opening, through the pedicle 18, into the vertebral body of vertebra 14, and through the bottom endplate of the vertebra 14. The passage 118 may continue through the top endplate of the vertebra 16 and out through the anterior cortical wall of the vertebra 16. The passage 118 may extend diagonally through the spinal joint 10 such that the passage emerges through the anterior wall of the vertebra 16 on the side laterally opposite the pedicle 18. The cannulated pedicle screw 96 may be threaded into passage 118 in the pedicle 18. The connection element 92 may be anchored by the screw 96, extend through the screw 96, and further extend through the passage 118 in the vertebrae 14, 16. Where the connection element 92 passes through the outer wall of vertebra 16, the stopper feature 100 may be added to anchor the connection element 92 to the vertebra 16.

The passage 118 may be formed using a minimally invasive, flexible, steerable drill (not shown). The drill may be steered with radiographic guidance. In alternative embodiments, the passage may be created using a stylet or another type of cutting or tunneling instrument. All or portions of the passage may even be formed by the connecting element itself. The passages through both vertebrae 14, 16 may be formed through a single posterior approach from the pedicle 18. Alternatively, the passage through the vertebra 14 may be formed through the pedicle 18 and the passage through the vertebra 16 may be formed through a separate approach, such as through an anterior opening in the vertebra 16.

The connection elements 94, 104, 106; the anchoring structures 98, 108, 110; and the stopper features 102, 112, 114, respectively may be implanted in a manner similar to the method described above for the implantation of connecting element 32. In this embodiment, the connection element 94 may extend from the anchoring structure 98 in pedicle 20, through the nucleus replacement 116, and through an anterior opening of the vertebral body 16 to be held in place by the stopper feature 102. The connection element 104 may extend from the anchoring structure 108 in pedicle 22, through the nucleus replacement 116, and through an anterior opening of the vertebral body 14 to be held in place by the stopper feature 112. The connection element 106 may extend from the anchoring structure 110 in pedicle 24, through the nucleus replacement 116, and through an anterior opening of the vertebral body 14 to be held in place by the stopper feature 114. The diagonal paths taken by connection elements 92, 94, 104, 106 may lend lateral stability to the joint 10 by limiting lateral translation between the vertebrae 14, 16 and by limiting lateral bending at the joint 10. The system 90 may also provide axial stability by limiting axial rotation of the vertebra 14 relative to the vertebra 16. Additionally, the connection elements 92, 94, 104, 106 may position the nucleus replacement 116 in a desired location, preventing migration or expulsion.

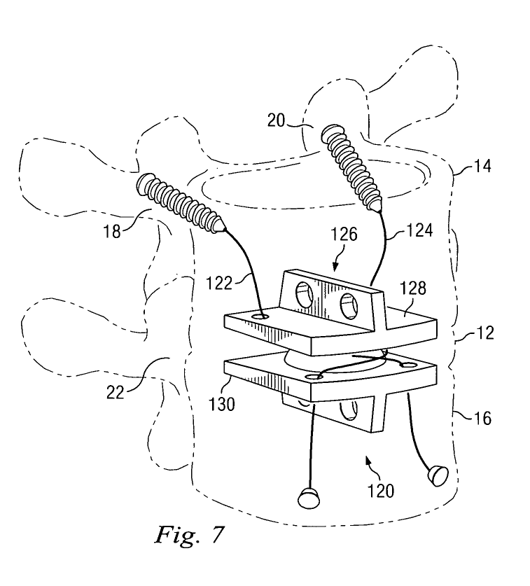

Referring now to FIG. 7, a vertebral joint stabilization system 120 may include connection elements 122, 124 which may be anchored to the pedicles of vertebra 14. The system 120 may be similar to the system 50 with certain differences to be described below. In this embodiment, all or a portion of the damaged tissue including the nucleus of disc 12 may be excised. The damaged disc 12 may be replaced by an intervertebral disc prosthesis 126 which may be selected from a variety of devices including any of the prostheses which have been described in U.S. Pat. Nos. 5,674,296; 5,865,846; 6,156,067; 6,001,130; 6,740,118 and in U.S. Patent Application Pub. Nos. 2002/0035400; 2002/0128715; and 2003/0135277; 2004/0225366 which are incorporated by reference herein. It is understood that in an alternative embodiment, the prosthesis may be an intervertebral fusion device. The nucleus replacements, the intervertebral disc prostheses, and the intervertebral fusion devices referenced above may be referred to as prosthetic devices, intervertebral joint prostheses, prosthetic implants, disc prostheses, or, artificial discs.

In this embodiment, the prosthesis 126 may be a implant similar to that described in U.S. Pat. No. 6,740,118 to Eisermann et al. The prosthesis 126 may include components in the form of an upper endplate 128 and a lower endplate 130. The prosthesis 126 may be implanted using an anterior, lateral, oblique, or any other implantation method known in the art.

The connection elements may be formed of a biocompatible synthetic material or graft material such as autograft or allograft tendon material with bone attached to each end. The connection element material may, for example, take the form of a woven cable or unwoven strands. The connection elements may be adapted to withstand at least some tensile and/or torsional forces and may slacken when submitted to compressive forces. Additionally, the connection elements may be adapted to withstand at least some compressive and moment forces. Solid wire or rods, for example, may withstand such forces. These connection elements may permit some amount of flexibility and buckling when subject to compressive and/or moment loads.

The connection elements 122, 124 may be implanted using a method similar to that described above in system 50 to supplement or stabilize the function of the prosthesis 126. In this embodiment, the connection elements 122, 124 may extend through the prosthesis 126 to prevent migration or expulsion of the prosthesis and to limit motion. Connection element 122 may be anchored to and extend through the pedicle 18 into the vertebral body of vertebra 14, through the upper endplate 128, and through the lower endplate 130. The connection element 122 may continue the vertebral body 16 and through a portion of the anterior wall of the vertebra 16 generally laterally opposite the pedicle 18. The connection element 124 may follow a similar, but contralateral path. The connection element 124 may be anchored to the pedicle 20 and extend from the pedicle 20 through the vertebral body of vertebra 14. The connection element 124 may further extend through the prosthesis 126, through the vertebral body of the vertebra 16, and through a portion of the anterior wall of the vertebra 16 laterally opposite the pedicle 20.

The connection elements 122, 124 may provide lateral stability to the prosthesis 126 by limiting lateral translation and lateral bending between the endplates 128, 130. For example, lateral bending to the patient's right side may place the connection element 124 into tension, thereby limiting the right side lateral bending. Similarly, lateral translation of the endplate 128 to the right relative to the endplate 130 may also place the connection element 124 into tension to limit the relative translation of the endplates. The connection elements 122, 124 may also provide axial stability to the prosthesis 126 and thereby the joint 10 by limiting axial rotation of the endplate 128 relative to the endplate 130. For example, axial rotation to the patient's right may place one or both of the connection elements 122, 124 into tension, thus restricting axial rotation.

In still another alternative embodiment, a stabilization system similar to system 30 may include connection elements anchored to the superior vertebral body 14a rather than the pedicles 18, 20. In this embodiment, the connection element may be anchored to a wall of the vertebral body 14a using anchoring devices such as suture anchors, elastic plugs, or knots. Thus, in this embodiment, both ends of the connection elements may be anchored to vertebral bodies rather than pedicles. Alternatively, the connection elements may be anchored within the vertebral body 14a to, for example, a bolus of cement. Connections to other posterior processes of the vertebra 14 without passage through the pedicles 18, 20 is also possible.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “upper,” and “lower,” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A system for stabilizing a vertebral joint comprising:
a first connection element adapted to extend through a first pedicle of a first vertebra, through at least a portion of a vertebral body of the first vertebra, and into an endplate of a second vertebra and
a second connection element adapted to extend through a second pedicle of a first vertebra, through at least a portion of the vertebral body of the first vertebra, and into the endplate of the second vertebra.

2. The system of claim 1 wherein the first connection element further extends into at least a portion of a vertebral body of the second vertebra.

3. The system of claim 1 wherein the first connection element further extends through an outer wall of a vertebral body of the second vertebra.

4. The system of claim 3 further comprising:
an anchoring component attached to the first connection element at the outer wall.

5. The system of claim 1 wherein the first connection element extends into a first portion of the endplate of the second vertebra laterally opposite from the first pedicle.

6. The system of claim 2 wherein the second connection element extend into a second portion of the endplate of the second vertebra laterally opposite from the second pedicle.

7. The system of claim 1 wherein the first connection element is formed of synthetic material.

8. The system of claim 1 wherein the first connection element is formed of graft tendon.

9. The system of claim 1 further comprising:
a first cannulated pedicle screw adapted to engage the first pedicle and receive the first connection element and
a second cannulated pedicle screw adapted to engage the second pedicle and receive the second connection element.

10. The system of claim 1 further comprising:
a prosthetic device adapted for insertion between the first and second vertebrae and further adapted to accept through passage of the first and second connection elements.

11. The system of claim 10 wherein the prosthetic device is a nucleus replacement device.

12. The system of claim 10 wherein the prosthetic device is an intervertebral disc prosthesis.

13. The system of claim 10 wherein the prosthetic device is adapted for fusing the first and second vertebrae.

14. The system of claim 1 wherein the first and second connection elements limit lateral bending of the first vertebra relative to the second vertebra.

15. The system of claim 1 wherein the first and second connection elements limit translation of the first vertebra relative to the second vertebra.

16. The system of claim 1 wherein the first and second connection elements limit axial rotation of the first vertebra relative to the second vertebra.

17. The system of claim 1 wherein the first connection element further extends through a pedicle of the second vertebra.

18. The system of claim 1 further comprising:
a third connection element adapted to extend through a pedicle of the second vertebra, through a vertebral body of the second vertebra, and into an endplate of the first vertebra.

19. A method of stabilizing a vertebral joint between first and second vertebrae, the method comprising:
creating a first passage through a first pedicle of the first vertebra and into a vertebral body of the first vertebra;
guiding a first connection element into the first passage and through the vertebral body of the first vertebra; and
guiding the first connection element into the second vertebra.

20. The method of claim 19 further comprising:
creating a second passage through a second pedicle of the first vertebra and into the vertebral body of the first vertebra;
guiding a second connection element into the second passage and through the vertebral body of the first vertebra;
guiding the second connection element into the second vertebra.

21. The method of claim 20 wherein the second connection element crosses the first connection element.

22. The method of claim 19 wherein the step of creating comprises drilling the first passage.

23. The method of claim 22 wherein the drilling is performed with a flexible drill.

24. The method of claim 19 wherein the step of creating the first passage is conducted under radiographic guidance.

25. The method of claim 19 further comprising:
anchoring the first connection element to an outer wall of the second vertebra.

26. The method of claim 19 further comprising:
adding a stopper feature to the first connection element to anchor the first connection element to the second vertebra.

27. The method of claim 19 further comprising:
inserting a cannulated pedicle screw into the first passage and
guiding the first connection element through the cannulated pedicle screw.

28. The method of claim 19 further comprising:
passing the first connection element through a prosthetic device, wherein the prosthetic device is positioned between the first and second vertebrae.

29. The method of claim 19 wherein the steps of creating and guiding are performed using minimally invasive instrumentation.

30. A system for stabilizing a vertebral joint comprising:
means for connecting a first pedicle of a first vertebra to a vertebral body of a second vertebra and
means for connecting a second pedicle of the first vertebra to the vertebral body of the second vertebra.

31. The system of claim 30 further comprising means for replacing at least a portion of a spinal disc.

32. A system for stabilizing a vertebral joint comprising:
a connection element adapted to extend through a first vertebral body, through a vertebral endplate of the first vertebral body, through a vertebral endplate of a second vertebral body, and through the second vertebral body.
